(19) United States
(12) Patent Application Publication
(10) Pub. No.: US 2011/0251676 A1
(43) Pub. Date: Oct. 13, 2011

(54) Sheath for Controlled Delivery of a Heart Valve Prosthesis

(75) Inventors: Fiachra Sweeney, Galway (IE); Paula McDonnell, Galway (IE); Brian Kelly, Galway (IE); Patrick Duane, Galway (IE); John MacNamara, Galway (IE); Frank Harewood, Galway (IE)

(73) Assignee: Medtronic Vascular, Inc., Santa Rosa, CA (US)

(21) Appl. No.: 12/758,272

(22) Filed: Apr. 12, 2010

(51) Int. Cl. A61F 2/84

(52) U.S. Cl. 623/1.23

(57) ABSTRACT

Apparatus and methods are disclosed for controlling deployment of a self-expanding support structure of a prosthetic valve that flares in a proximal direction upon implantation in vivo. A tubular delivery sheath having a side opening that proximally extends within a side wall thereof is used to deploy the prosthetic valve with the self-expanding support structure in a controlled manner. The prosthetic valve is distally advanced within a lumen of the delivery sheath with the self-expanding support structure held in a compressed delivery configuration within the delivery sheath lumen. The self-expanding support structure of the prosthetic valve is aligned with the side opening of the delivery sheath and the prosthetic valve is rotated relative to the delivery sheath whereby the self-expanding support structure is laterally released from the delivery sheath lumen through the side opening to gradually transition from the compressed delivery configuration to a flared deployed configuration.

### FIELD OF THE INVENTION

The invention relates generally to delivery systems for deploying a prosthetic heart valve in a non-coronary bypass procedure. More particularly, the invention relates to a delivery sheath for controlling deployment of a self-expanding support structure of the prosthetic heart valve.

### BACKGROUND OF THE INVENTION

A wide range of medical treatments are known that utilize “endoluminal prostheses.” As used herein, endoluminal prostheses are intended to mean medical devices that are adapted for temporary or permanent implantation within a body lumen, including both naturally occurring and artificially made lumens. Examples of lumens in which endoluminal prostheses may be implanted include, without limitation: cardiac structures and valves, arteries, such as those located within the arteries, veins gastrointestinal tract, biliary tract, urethra, trachea, hepatic and cerebral shunts, and fallopian tubes.

Stent prostheses are known for implantation within a body lumen for providing artificial radial support to the wall tissue that defines the body lumen. To provide radial support to a blood vessel, such as one that has been widened by a percutaneous transluminal coronary angioplasty, commonly referred to as “angioplasty,” “PTA” or “PTCA”, a stent may be implanted in conjunction with the procedure. Under this procedure, the stent may be collapsed to an insertion diameter and inserted into the vasculature at a site remote from the diseased vessel. The stent may then be delivered to the desired treatment site within the affected vessel and deployed, by self-expansion or radial expansion, to its desired diameter for treatment.

Recently, flexible prosthetic valves supported by stent-like structures that can be delivered percutaneously using a catheter-based delivery system have been developed for heart and venous valve replacement. These prosthetic valves may include either self-expanding or balloon-expandable stent structures with valve leaflets attached to the interior of the stent structure. The prosthetic valve can be reduced in diameter, by crimping onto a balloon catheter or by being contained within a sheath component of a delivery catheter, and advanced through the venous or arterial vasculature. Once the prosthetic valve is positioned at the treatment site, for instance within an incompetent or diseased native valve, the stent structure may be expanded to hold the prosthetic valve firmly in place. One embodiment of a stented prosthetic heart valve is disclosed in WO 2008/035337 A2 to Tuval et al. entitled “Fixation Member for Valve” (hereinafter referred to as “the Tuval et al. publication”), which is incorporated by reference herein in its entirety.

When a prosthetic valve is deployed at the treatment site, fundamental concerns are that (a) the prosthesis be deployed as precisely as possible and (b) that the deployment be controlled so as not to damage any surrounding structures, particularly where the prosthetic valve is used to replace an insufficient, diseased or incompetent heart valve. However, providing controlled deployment of a prosthetic valve to assure accurate positioning thereof may be difficult due to the complexities in the anatomy and an initial deployment of the prosthetic valve may result in a less than optimal positioning or, even worse, an inoperable positioning. Further some prosthetic heart valves have self-expanding support structures with proximal portions that flare outward to be subsequently positioned within the sinuses of the incompetent heart valve. For example, FIG. 1 illustrates an embodiment of a heart valve prosthesis 100 disclosed in the Tuval et al. publication that when delivered via a transapical approach includes a distal fixation member 114 configured to be positioned in a downstream artery, such as the ascending aorta, and shaped to define three proximal engagement arms 122 that are configured to be positioned at least partially within respective natural sinuses. Proximal engagement arms 122 flare outward and are described in the Tuval et al. publication as being “generally upwardly concave” or “concave in a downstream direction” when implanted. Prosthesis 100 further includes a proximal fixation member 112 that seats within the native valve and extends partially into the left ventricle when the native valve is the aortic valve. Proximal engagement arms 122 of prosthesis 100 are held in compressed delivery configuration when loaded within a conventional delivery sheath or trocar prior to deployment. When deployed from a distal end of the delivery sheath or trocar the engagement arms 122 tend to concurrently spring open upon clearing the distal end in an uncontrolled manner, which may result in prosthesis 100 having a less than optimal or inoperable position within the native valve or damaging surrounding structures. A more gradual and controlled release of a prosthetic heart valve having a support structure with proximal engagement arms 122 is desirable to ensure slow, controlled release of the arms, avoiding contact with surrounding structures such as the ascending aorta and assure optimal positioning of the engagement arms within the natural sinuses. As such there is a need in the art for a prosthetic valve delivery system that permits controlled deployment of a prosthetic valve having a self-expanding support structure in which at least a proximal portion of the support structure flares or spreads outward.

### BRIEF SUMMARY OF THE INVENTION

Embodiments hereof are directed to apparatus and methods for controlling deployment of a self-expanding support structure of a prosthetic valve that flares in a proximal direction upon implantation in vivo. A tubular delivery sheath in accordance with embodiment hereof includes at least one side opening that proximally extends within a side wall of the delivery sheath from a distal end thereof. The prosthetic valve with the self-expanding support structure is deployed in a controlled manner through the delivery sheath side opening. More particularly, the prosthetic valve is distally advanced within a lumen of the delivery sheath with the self-expanding support structure held in a compressed delivery configuration within the delivery sheath lumen. The self-expanding support structure of the prosthetic valve is aligned with the side opening of the delivery sheath and the prosthetic valve is rotated relative to the delivery sheath whereby the self-expanding support structure is laterally released from the delivery sheath lumen through the side opening to gradually transition from the compressed delivery configuration to a flared deployed configuration.

A delivery sheath in accordance with another embodiment hereof may include a plurality of side openings that proximally extend within a side wall thereof to accommodate simultaneous controlled release of a plurality of self-expanding support structures of the prosthetic valve each of which flares in a proximal direction upon implantation in vivo. The prosthetic valve is distally advanced within a lumen of the delivery sheath with the self-expanding support structures held in a compressed delivery configuration within the delivery sheath lumen. Each of the self-expanding support structures of the prosthetic valve is aligned with a respective side opening of the delivery sheath and the prosthetic valve is rotated and withdrawn relative to the delivery sheath whereby the self-expanding support structures are simultaneously laterally released from the delivery sheath lumen through the respective side openings to gradually transition from the compressed delivery configuration to a flared deployed configuration.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a schematic illustration of a prior art heart valve prosthesis in a deployed configuration that may be collapsed or compressed for delivery in accordance with embodiments hereof.

FIG. 2 is a perspective view of a delivery sheath in accordance with an embodiment hereof.

FIG. 3 is a sectional side view of the delivery sheath of FIG. 2 with the heart valve prosthesis of FIG. 1 loaded within a delivery catheter and positioned therein.

FIG. 4 is a perspective view of a delivery system in accordance with another embodiment hereof.

FIGS. 5-7 illustrate a method of implanting the heart valve prosthesis of FIG. 1 within a native valve via a transapical approach with the delivery sheath of FIG. 2.

FIGS. 8-10 illustrate a method of controlling the deployment of a self-expanding support structure having flared proximal portions with a delivery sheath in accordance with another embodiment hereof.

FIGS. 11 and 12 illustrate a method of controlling the deployment of a self-expanding support structure having flared proximal portions with a delivery sheath in accordance with another embodiment hereof.

FIG. 13 is a side view of a distal end of a delivery sheath in accordance with an alternate embodiment hereof.

FIGS. 14 and 15 illustrate a method of controlling the deployment of a self-expanding support structure having flared proximal portions with a delivery sheath in accordance with another embodiment hereof.

FIGS. 16-19 are side views of a distal end of a delivery sheath in accordance with alternate embodiments hereof.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the in the context of heart valve replacement, the invention may also be used for stent or valve replacement in other body passageways where it is deemed useful. Furthermore, there is no intention to be bound by any expressed or implied theory presented in the preceding technical field, background, brief summary or the following detailed description.

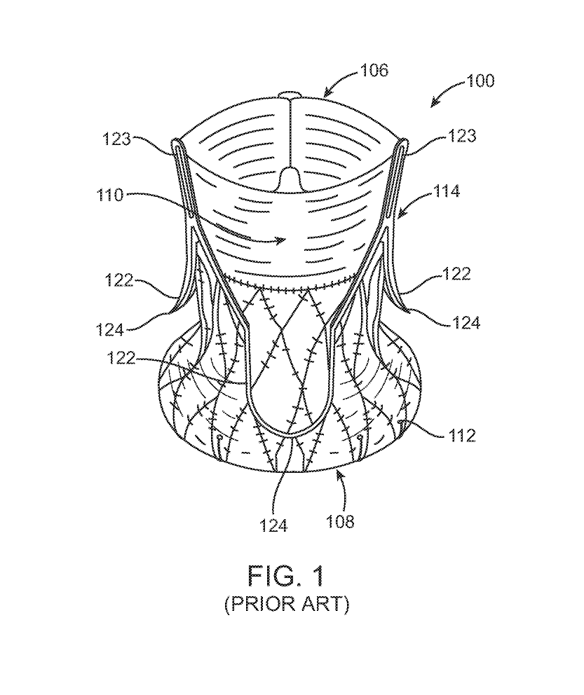

As noted above, FIG. 1 is a schematic illustration of a prior art heart valve prosthesis 100 in a deployed configuration. In addition to proximal and distal fixation members 112, 114, heart valve prosthesis 100 includes a valve component 110 that is configured to collapse inwardly, i.e., towards a longitudinal axis of heart valve prosthesis 100, during diastole in order to inhibit retrograde blood flow and to open outwardly during systole to allow blood flow through heart valve prosthesis 100. Proximal and distal fixation members 112, 114 are collapsible and made of a material having resiliency or shape memory characteristics in order to return heart valve prosthesis 100 to the deployed configuration shown in FIG. 1 upon release from a delivery device. The structure and operation of heart valve prosthesis 100 are more fully described in the Tuval et al. publication, which was previously incorporated by reference herein in its entirety.

Heart valve prosthesis 100 may be described as having a distal or upstream end 106 and a proximal or downstream end 108, wherein “distal” and “proximal” are relative to a clinician delivering the heart valve prosthesis via a transapical approach and “upstream” and “downstream” are relative to a direction of blood flow when the heart valve prosthesis is properly implanted in vivo. Engagement arms 122 of distal fixation member 114 are generally u-shaped and proximally extend between strut supports 123 of distal fixation member 114 to be positioned between the distal and proximal ends 106, 108 of heart valve prosthesis 100. In addition proximal ends 124 of engagement arms 122 may be described as being radially spaced or flared from the remainder of distal fixation member 114 and engagement arms 122 may be described as having a curved profile in the deployed/implanted configuration in order to engage the sinuses. Engagement arms 122 may also be described to be flared in a proximal direction or proximally flared in a deployed configuration as proximal ends 124 of engagement arms 122 are radially spaced from the remainder of distal fixation member 114 and substantially extend in the proximal direction of the valve prosthesis when the valve prosthesis is implanted. When compressed for delivery within a conventional delivery sheath or trocar, engagement arms 122 will somewhat straighten against proximal fixation member 112 such that when released from the distal end of the conventional delivery sheath or trocar, engagement arms 122 will substantially simultaneously and suddenly spring back to their curved configuration, which may result in a suboptimal positioning of heart valve prosthesis 100 or damage to surrounding structures.

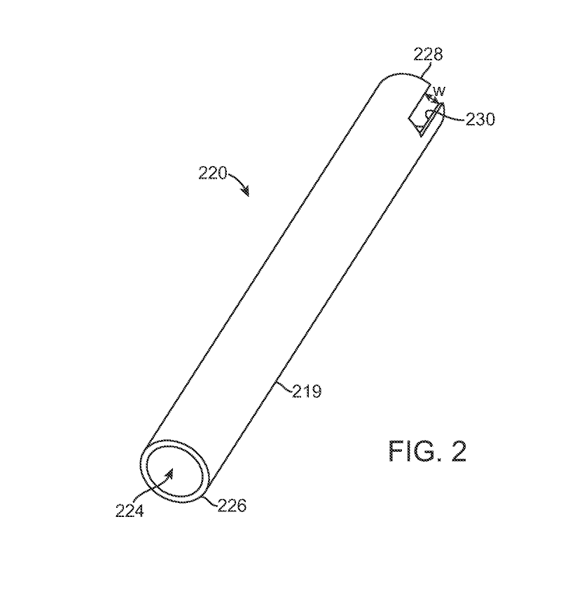

FIG. 2 is a perspective view of a delivery sheath or trocar 220 in accordance with an embodiment hereof. Delivery sheath 220 has a tubular or cylindrical body portion 219 defining a delivery lumen 224 between a proximal end 226 and a distal end 228 thereof. Delivery lumen 224 is sized to slidably receive a delivery catheter or other delivery device therein, an embodiment of which will be described in more detail below. A side opening or slot 230 is formed through a side wall of body portion 219 to extend proximally from delivery sheath distal end 228. In the embodiment of FIG. 2, side opening 230 has a substantially rectangular shape that is defined on three sides by body portion 219 and has a width W that is open to delivery sheath distal end 228. Side opening 230 is sized to allow controlled lateral or transverse deployment of engagement arms 122 of heart valve prosthesis 100 there through as described in more detail below. In an embodiment, the delivery sheath is a semi-rigid to rigid structure, with the portion surrounding the side opening having enough strength to allow controlled opening of the engagement arms of the valve prosthesis, as discussed below. In embodiments hereof, the delivery sheath may be constructed of various polymeric materials such as polyether ether ketone (PEEK), metallic materials such as stainless steel, reinforced polymers such as a polyimide with a braided metallic reinforcing layer, or modified metals. In embodiments hereof, the portion of the delivery sheath surrounding the side opening may be thinner than the remainder of the delivery sheath, profiled and/or tapered to pass between the engagement arms 122 and the downstream end 108 of heart valve prosthesis 100.

In embodiments hereof, the thickness of a wall of the delivery sheath depends on the materials selected for the tube from which the sheath is formed, for e.g., a metal tube may be as thin as 0.05 mm, whereas a polymeric tube could be as thick as 1.5 mm. Further the internal diameter of the delivery sheath will depend on the valve prosthesis design and dimensions to be delivered therefrom such that in certain embodiments an internal diameter of the delivery sheath may be in the range of 7 mm to 10 mm. The overall length of the delivery sheath may be in the range of 100 mm to 200 mm depending on the application in which it is to be used.

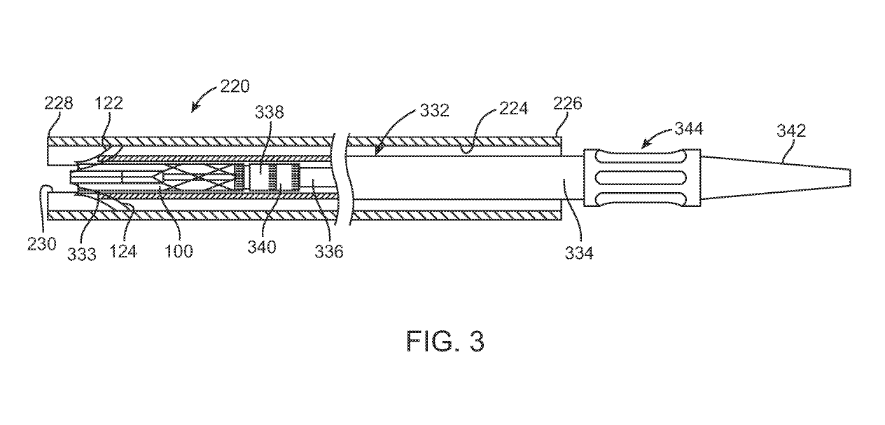

FIG. 3 is a sectional side view of delivery sheath 220 with a delivery catheter 332 positioned within delivery sheath lumen 224, wherein heart valve prosthesis 100 is shown loaded within delivery catheter 332. Delivery catheter 332 includes an outer tubular member 334 attached at a proximal end to a handle 342 with an inner tubular member 336 coaxially positioned therein that is coupled at a proximal end to a rotatable delivery knob 344 and attached at a distal end to a connector 340. Proximal fixation member 112 of heart valve prosthesis 100 is detachably coupled to a device holder 338, which is threadably connected to connector 340 of inner tubular member 336. Heart valve prosthesis 100 extends from a distal end 333 of outer tubular member 334 a sufficient distance to allow engagement arms 122 to be radially positioned between outer tubular member 334 and delivery sheath 220 such that engagement arms 122 are held or compressed in a delivery configuration by delivery sheath 220. The structure and operation of delivery catheter 332 are more fully described in the Tuval et al. publication, which was previously incorporated by reference herein in its entirety.

In order to control deployment of engagement arms 122 of heart valve prosthesis 100, delivery catheter 332 is distally advanced relative to delivery sheath 220 in order to laterally align proximal ends 124 of engagement arms 122 with side opening 230. Delivery catheter 332/heart valve prosthesis 100 are then rotated relative to delivery sheath 220 to allow a respective engagement arm 122 to transversely slide through side opening 230 and thereby gradually or slowly, transition from the compressed delivery configuration to a proximally flared state, i.e., a flared deployed configuration. Continued relative rotation of delivery catheter 332/heart valve prosthesis 100 relative to delivery sheath 220 permits controlled consecutive or sequential deployment of the remaining engagement arms 122 in a like manner.

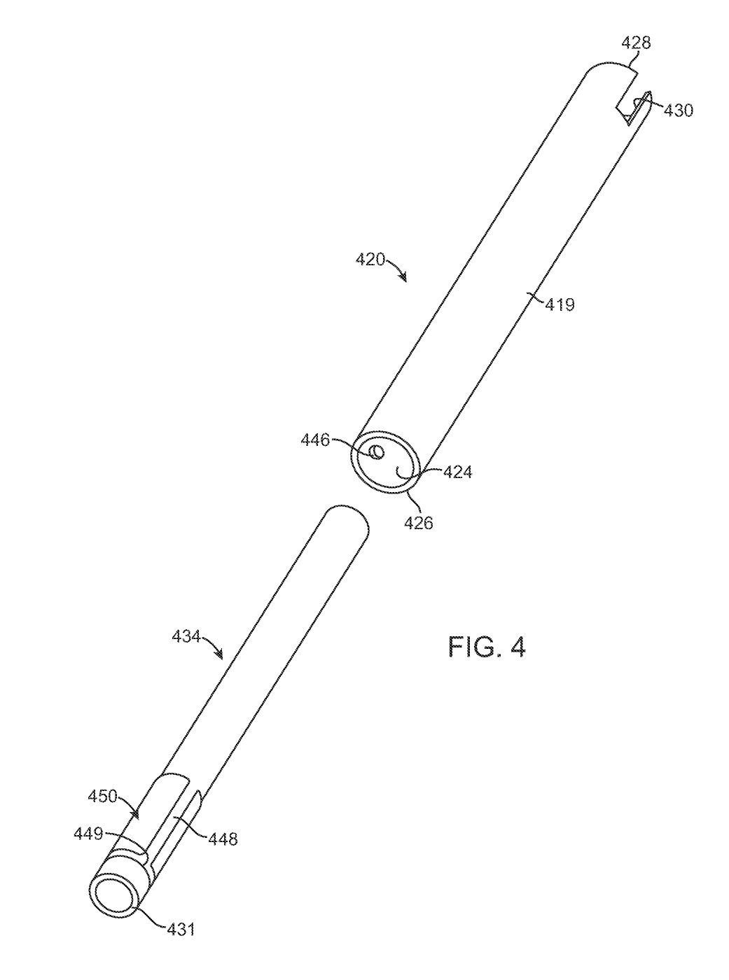

FIG. 4 is a perspective view of a delivery sheath or trocar 420 in accordance with another embodiment hereof. Delivery sheath 420 has a tubular or cylindrical body portion 419 defining a delivery lumen 424 between a proximal end 426 and a distal end 428 thereof. Delivery lumen 424 is sized to slidably receive a delivery catheter or other delivery device therein of which an outer tubular member 434 is shown in FIG. 4. A side opening or slot 430, which is substantially similar to side opening 230 described above, is formed through a side wall of tubular body portion 419 and proximally extends from delivery sheath distal end 428. Side opening 430 is sized to allow controlled deployment of engagement arms 122 of heart valve prosthesis 100, or other similar self-expanding support structures, as described above.

Delivery sheath 420 differs from delivery sheath 220 in that delivery sheath 420 includes a pin 446 projecting within delivery lumen 424. Pin 446 is positioned near delivery sheath proximal end 426 and is sized to be slidably received within a T-shaped groove or slot 448 that is formed in an outer surface of outer tubular member 434 near a proximal end 431 thereof. In an embodiment, T-shaped groove 448 is formed within a sleeve 450 that surrounds and is attached to outer tubular member 434. In order to couple the delivery catheter together with delivery sheath 420, pin 446 is proximally slid within T-shaped groove 448 until pin 446 reaches juncture 449 at which point delivery sheath 420 is rotated relative to outer tubular member 434 to slide pin 446 within the circumferential portion of groove 448, which thereby “locks” a longitudinal position of outer tubular member 434 relative to delivery sheath 420. In this manner, pin 446 and T-shaped groove 448 are used to ensure rotational alignment between outer tubular member 434 and delivery sheath 420 such that the engagement arms 122 align with side openings 430. In addition, this arrangement prevents relative longitudinal movement between outer tubular member 434, i.e., the delivery catheter, and delivery sheath 420 as the components are being tracked to and positioned across an incompetent valve, which thereby prevents premature or unintended deployment of heart valve prosthesis engagement arms 122, or other similar self-expanding support structures, through delivery sheath side opening 430. In another embodiment, pin 446 may be, for example, spring loaded to engage with indentations in the circumferential portion of groove 448, which would aid in the alignment and controlled deployment of the engagement arms 122 from side openings 430.

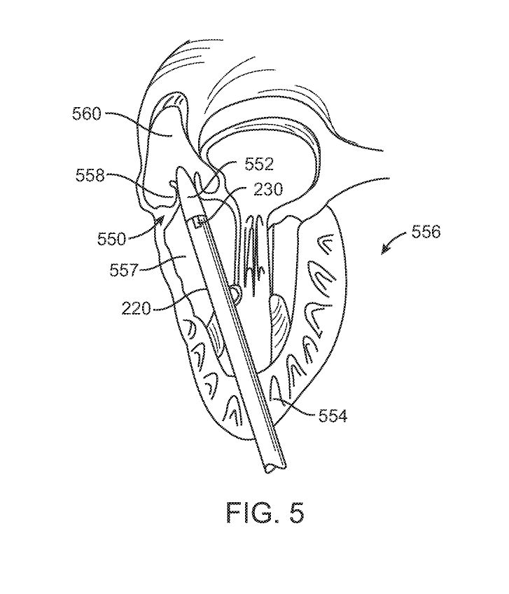

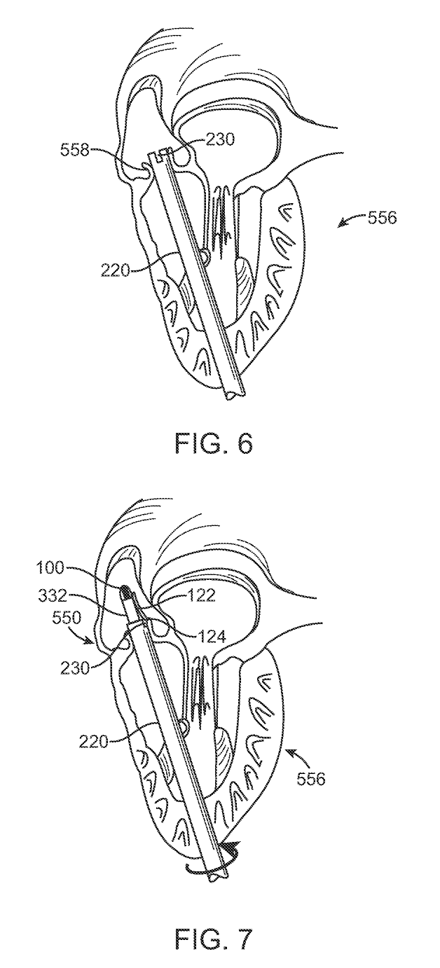

FIGS. 5-7 illustrate a method of implanting heart valve prosthesis 100 within a diseased or damaged aortic valve 550 via a transapical approach utilizing delivery sheath 220. As shown in FIG. 5, delivery sheath 220, which has been placed over a dilator 552, has been inserted through the apex 554 of heart 556, and advanced through left ventricle 557 until a distal end of dilator 552 passes native aortic valve leaflets 558. As would be understood by one of ordinary skill in the art, apex 554 may be punctured using a standard Seldinger technique, and a guidewire may be advanced into the ascending aorta 560. Delivery sheath 220 may then be backloaded onto the guidewire and tracked or advanced thereover into the ascending aorta 560. Delivery sheath 220 is advanced beyond aortic valve 550 such that a proximal end of side opening 230 is located distal of native aortic valve leaflets 558 and dilator 552 is removed, as shown in FIG. 6. Delivery catheter 332 with heart valve prosthesis 100 loaded therein as described above with reference to FIG. 3 is longitudinally advanced relative to delivery sheath 220 until proximal ends 124 of engagement arms 122 are laterally aligned with side opening 230 of delivery sheath 220 and distal to native valve leaflets 558. Once so aligned subsequent rotation of delivery catheter 332 and heart valve prosthesis 100 relative to delivery sheath 220 allows each engagement arm 122 to be laterally or transversely released through side opening 230 so as to permit consecutive controlled deployment of engagement arms 122. More particularly, as heart valve prosthesis 100 is rotated relative to delivery sheath 220, a first engagement arm 122 slides through side opening 230 to return to its deployed/flared state, as shown in FIG. 7. Continued rotation of heart valve prosthesis 100 relative to delivery sheath 220 allows a second engagement arm 122 and then a third engagement arm 122 to slide sequentially through side opening 230 and thereby consecutively return to their deployed/flared state in a controlled manner. Once engagement arms 122 have been released from delivery sheath 220, delivery catheter 332 with heart valve prosthesis 100 are withdrawn proximally and rotated such that engagement arms 122 sit within the sinuses. Once position is confirmed, for e.g., using fluoroscopy, release of the prosthesis 100 is completed by withdrawing the delivery sheath 220 until proximal fixation member 112 is released. Delivery sheath 220 is then retracted from the patient and heart valve prosthesis 100 is deployed from delivery catheter 332 as discussed in detail in the Tuval et al. publication.

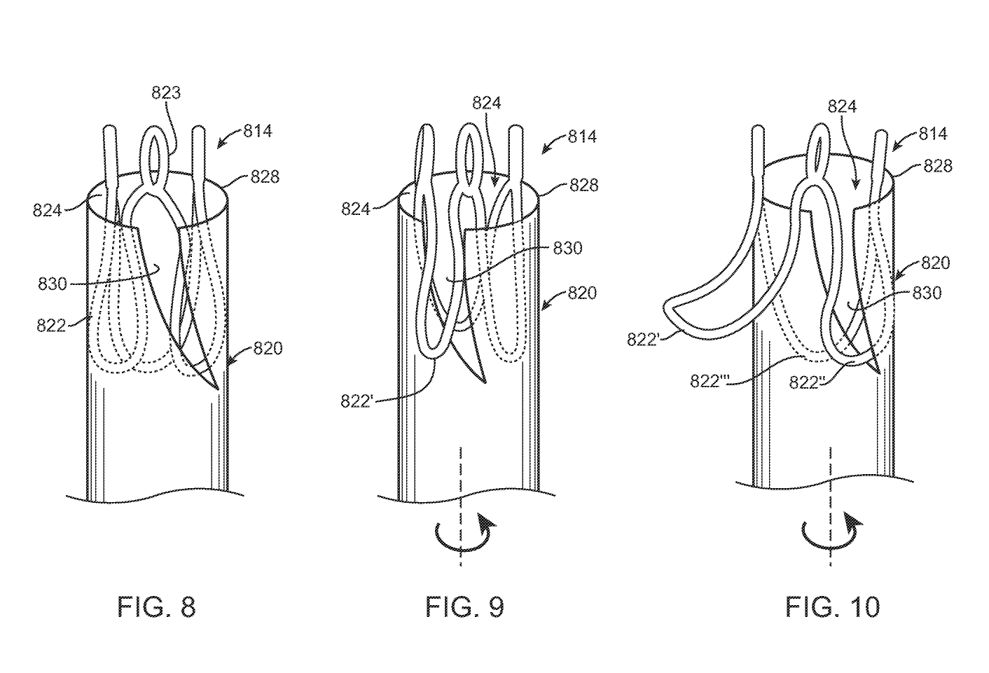

FIGS. 8-10 illustrate a method of controlling the deployment of a self-expanding support structure 814 having flared proximal portions 822 with a delivery sheath 820 in accordance with another embodiment hereof. It would be understood by one of ordinary skill in the art that self-expanding support structure 814 may be utilized to support a prosthetic heart valve such as prosthetic heart valve 100 described above. In a deployed configuration proximal portions 822 of self-expanding support structure 814 may be described as flared in a proximal direction or proximally flared as proximal ends of proximal portions 822 are radially spaced from the remainder of self-expanding support structure 814 and are intended to substantially extend in the proximal direction of the prosthetic heart valve when implanted. Similar to previous embodiments, delivery sheath 820 has a distal end 828 and a proximal end (not shown) with a delivery lumen 824 that extends therebetween. A wedge or wave-like shaped side opening or slot 830 proximally extends within a side wall of delivery sheath 820 from distal end 828 and is sized to permit flared proximal portions 822 of support structure 814 to transversely slide there through when delivery sheath 820 is rotated. In FIG. 8 self-expanding support structure 814 is shown with distal ends of strut supports 823 projecting slightly from delivery sheath distal end 828 and with proximal portions 822 compressed within delivery lumen 824. FIG. 9 illustrates a first proximal portion 822′ of self-expanding support structure 814 engaging and sliding through side opening 830 as delivery sheath 820 is rotated to thereby gradual resuming its flared shape. FIG. 10 illustrates a second proximal portion 822″ of self-expanding support structure 814 engaging and sliding through side opening 830 as delivery sheath 820 is further rotated with first proximal portion 822′ having achieved its deployed configuration, i.e., its fully flared shape. Continued rotation of delivery sheath 820 relative to self-expanding support structure 814 permits a final proximal portion 822′″ to slide through side opening 830 and to return to its flared configuration at which point self-expanding support structure 814 is fully released from delivery sheath 820.

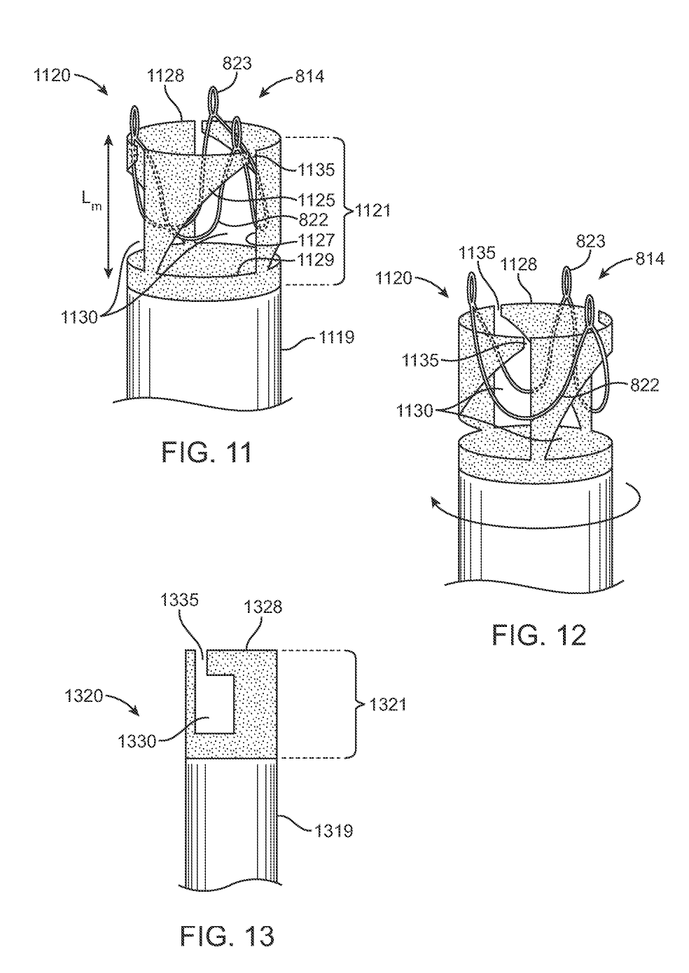

FIGS. 11 and 12 illustrate a method of controlling the deployment of self-expanding support structure 814 having flared proximal portions 822 with a delivery sheath 1120 in accordance with another embodiment hereof. Similar to previous embodiments, delivery sheath 1120 has a distal end 1128 and a proximal end (not shown) with a delivery lumen that extends therebetween. Delivery sheath 1120 has three side openings 1130 circumferentially spaced within a side wall of the delivery sheath near distal end 1128, wherein each side opening 1130 includes a right triangle shaped portion that is defined by a side hypotenuse segment 1125, a side leg segment 1127 and a base leg segment 1129. Base leg segments 1129 of side openings 1130 are of a length and are circumferentially spaced one from another to permit respective flared proximal portions 822 of support structure 814 to simultaneously laterally extend there through when delivery sheath 1120 is longitudinally translated relative to support structure 814 as shown in FIG. 11. With reference to each triangular shaped portion of side opening 1130, side hypotenuse segment 1125 and side leg segment 1127 distally extend toward each other from base leg segment 1129 and are spaced from each other by a narrow channel portion 1135 of side opening 1130 that proximally extends between delivery sheath distal end 1128 and the triangular shaped portion of side opening 1130. Narrow channel portions 1135 are sized and circumferentially positioned such that no portion of support structure 814, particularly strut supports 823, passes through narrow channel portions 1135 when delivery sheath 1120 is longitudinally positioned relative to support structure 814 as shown in FIG. 11. As such self-expanding support structure 814 is only partially released from the delivery sheath lumen. In this manner, only flared proximal portions 822 of support structure 814 have been laterally released through the triangular shaped portion of side openings 1130 from the delivery sheath lumen and if a clinician is unsatisfied with their initial deployment, delivery sheath 1120 may be longitudinally translated in a reverse direction relative to support structure 814 to recapture flared proximal portions 822. Thus advancing or retracting delivery sheath 1120 relative to self-expanding support structure 814 (as represented by arrow L_{M} in FIG. 11) allows simultaneous controlled release of proximal portions 822 as well as recapture thereof when necessary or desired. Once proximal portions 822 have been released from delivery sheath 1120 in a satisfactory manner, rotation of delivery sheath 1120 relative to self-expanding support structure 814 allows simultaneous controlled release of the remainder of self-expanding support structure 814, i.e., strut supports 823, through respective narrow channel portions 1135, as shown in FIG. 12, whereby self-expanding support structure 814 is fully released from delivery sheath 1120. In an embodiment, the rotation required to release all three support structures at once is approximately 60°.

Side openings 1130 of delivery sheath 1120 are formed within a side wall of a distal tubular segment 1121 of delivery sheath 1120. Distal tubular segment 1121 may be made of a polymeric or metallic material, such as a tube of braided polyimide or stainless steel, that has sufficient strength to contain self-expanding support structure 814 within the delivery sheath lumen without deflecting or deforming whereas the remaining body portion or proximal segment 1119 of delivery sheath 1120 may be made of a more flexible polymeric such as PEEK or polyamide, or a metallic material such as stainless steel.

FIG. 13 is a side view of a distal portion of a delivery sheath 1320 in accordance with an alternate embodiment hereof. Delivery sheath 1320 has a distal tubular segment 1321 of a first material and a tubular body portion or proximal segment 1319 of a second material, wherein the first material may be stronger/stiffer than the second material similar to the embodiment described with reference to FIGS. 11 and 12. A side wall of distal tubular segment 1321 defines a side opening 1330 that is rectangular shaped and substantially closed on four sides except for a narrow channel 1335 that is open to delivery sheath distal end 1328 such that side opening 1330 is spaced from distal end 1320 by narrow channel 1335. When delivery sheath 1320 is longitudinally translated relative to a self-expanding support structure, such as self-expanding support structure 814 shown above, a flared proximal portion of the self-expanding support structure may laterally or transversely extend through side opening 1330 without any remaining portion of the self-expanding support structure being released from the delivery sheath lumen. In this manner, if a clinician is unsatisfied with the initial deployment of the self-expanding support structure, delivery sheath 1320 may be longitudinally translated in a reverse direction relative to the support structure to recapture the released flared proximal portion. Once the flared proximal portion of the self-expanding support structure has been released from delivery sheath 1320 in a satisfactory manner, rotation of delivery sheath 1320 relative to the self-expanding support structure allows controlled release of that portion of the self-expanding support structure through respective narrow channel 1335. Continued rotation of delivery sheath 1320 relative to the self-expanding support structure will sequentially release the remaining flared proximal portions of the support structure through side opening 1330 in a gradual and controlled fashion. In another embodiment, a side wall of distal tubular segment 1321 may define three side openings 1330 that are of a length and circumferential spacing to permit respective flared proximal portions of a self-expanding support structure to simultaneously extend transversely there through when delivery sheath 1320 is longitudinally translated relative to the self-expanding support structure, as similarly described with reference to the embodiment of FIGS. 11-12.

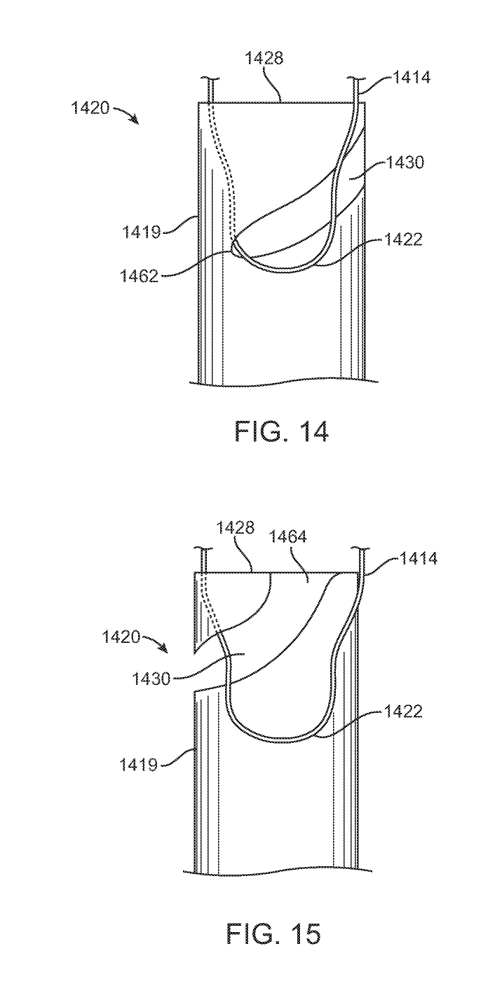

FIGS. 14 and 15 illustrate a method of controlling the deployment of a self-expanding support structure 1414 having flared proximal portions 1422 with a delivery sheath 1420 in accordance with another embodiment hereof. Similar to previous embodiments, delivery sheath 1420 has a distal end 1428 and a proximal end (not shown) with a delivery lumen that extends therebetween. Delivery sheath 1420 has a spiral opening or channel 1430 that is defined within a side wall of tubular body portion 1419 to wind there around from a closed proximal end 1462 to an open distal end 1464 at delivery sheath distal end 1428. In an embodiment, spiral channel 1430 may make one or more complete turns about tubular body portion 1419 before ending at delivery sheath distal end 1428. In use to control deployment of self-expanding support structure 1414, a delivery catheter or other delivery device with a valve prosthesis having support structure 1414 loaded therein (as similarly described above with reference to the embodiment of FIG. 3) is longitudinally advanced relative to delivery sheath 1420 until flared proximal portions 1422 are laterally aligned with closed proximal end 1462 of spiral channel 1430. Once so aligned subsequent rotation of the delivery catheter and heart valve prosthesis relative to delivery sheath 1420 allows each flared proximal portion 1422 to be laterally released in a sequential or consecutive manner through spiral channel 1430 so as to permit controlled deployment of self-expanding support structure 1414. More particularly, as self-expanding support structure 1414 is rotated relative to delivery sheath 1420, a first flared proximal portion 1422 slides within spiral channel 1430 to gradually return to its deployed/flared state as it travels along spiral channel 1430 and is released from open distal end 1464. A second flared proximal portion 1422 and then a third flared proximal portion 1422 will slide consecutively through spiral channel 1430 with continued rotation of self-expanding support structure 1414 relative to delivery sheath 1420 and thereby sequential return of each of the flared proximal portions to its deployed/flared state in a controlled manner is achieved. In an embodiment, spiral channel 1430 is of a length that permits subsequent flared proximal portions 1422 of self-expanding support structure 1414 to enter spiral channel 1430 at closed proximal end 1462 prior to release of the preceding flared proximal portion 1422 from open distal end 1464 such that more than one flared proximal portion 1422 may be sliding along spiral channel 1430 as delivery sheath 1420 is rotated. In another embodiment, the spiral channel may be longer and have a more gradual spiral angle or wider pitch to provide a more gradual release of the flared proximal portions.

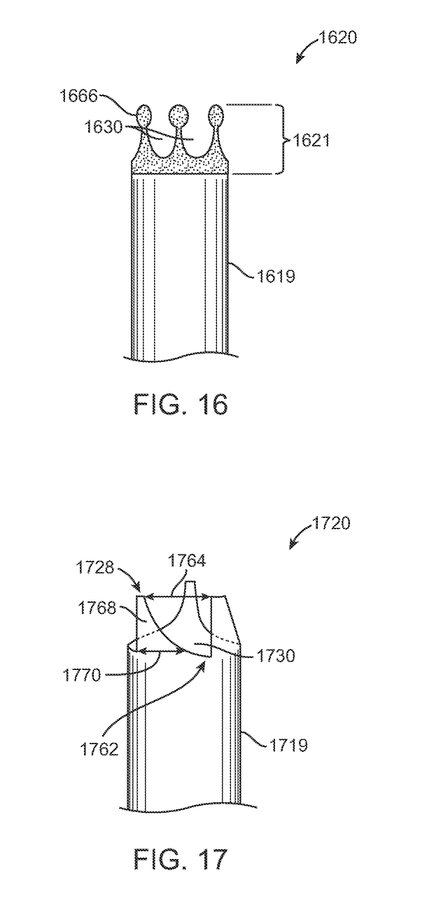

FIGS. 16-19 are side views of a distal end of a delivery sheath in accordance with alternate embodiments hereof. FIG. 16 illustrates a delivery sheath 1620 having a distal tubular segment 1621 of a first material attached to a tubular body portion or proximal segment 1619 of a second material similar to the embodiments of FIGS. 11-13 described above. Distal tubular segment 1621 has a crown-shape with bulbous-topped projections 1666 that define side openings 1630 therebetween. When a valve prosthesis having a self-expanding support structure, such as any of the support structures previously discussed above with flared proximal portions or engagement arms, is loaded with the delivery lumen of delivery sheath 1620, projections 1666 hold the flared proximal portions or engagement arms in a compressed configuration as the valve prosthesis is longitudinally translated relative to delivery sheath 1620 in order to bring the flared proximal portions into alignment with side openings 1630. Subsequent rotation of the valve prosthesis relative to delivery sheath 1620 allows simultaneous gradual release of the flared proximal portions transversely through respective side openings 1630 such that the flared proximal portions return to their flared deployed configuration in a controlled manner.

FIGS. 17-19 illustrate delivery sheaths 1720, 1820 and 1920 with similar features to one or more of the preceding embodiments and only the variation in side openings will be further described. In the embodiment of FIG. 17, delivery sheath 1720 includes a plurality of quadrant shaped side openings 1730, i.e., a side opening having the shape of a quarter-circle, that are circumferentially spaced within a side wall of tubular body portion 1719 about delivery sheath distal end 1728. Each of the quadrant-shaped openings 1730 distally widens from a proximal point 1762 to an open distal end 1764 at delivery sheath distal end 1728. Formation of quadrant-shaped side openings 1730 in tubular body portion 1719 leaves deployment guides 1768 between adjacent side openings 1730. Deployment guides 1768 have wide proximal base portions 1770 that resist a deflection force of a self-expanding support structure of a valve prosthesis when the valve prosthesis is loaded with the delivery lumen of delivery sheath 1720 prior to deployment. With reference to deployment of heart valve prosthesis 100 shown in FIG. 1, rotation of delivery sheath 1720 relative to heart valve prosthesis 100 allows simultaneous gradual release of engagement arms 122 transversely through respective side openings 1730 such that engagement arms 122 return to their flared configuration in a controlled manner. More particularly, each of deployment guides 1768 slides behind a respective engagement arm 122 of heart valve prosthesis 100 as delivery sheath 1720 is rotated to gradually release engagement arms 122 from the delivery sheath lumen laterally through side openings 1730 so that the engagement arms 122 may simultaneously return to their flared shapes in a controlled fashion.

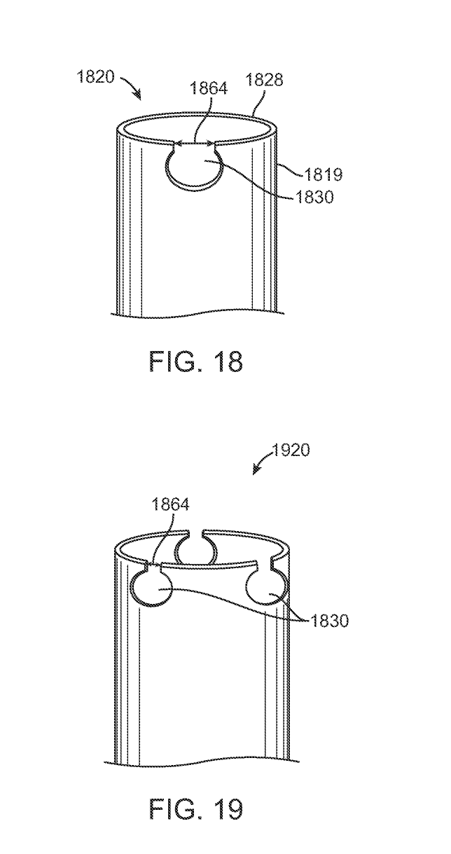

In the embodiment of FIG. 18, delivery sheath 1820 includes a side opening 1830 that is shaped to substantially match the rounded profile of proximal end 124 of engagement arm 122 of heart valve prosthesis 100. The circular shape of side opening 1830 is defined within a side wall of tubular body portion 1819 with an open distal end portion 1864 of between 10° to 60° extending along distal end 1828 of delivery sheath 1820. With reference to deployment of heart valve prosthesis 100 shown in FIG. 1, heart valve prosthesis 100 is longitudinally translated relative to delivery sheath 1820 to align proximal ends 124 of engagement arms 122 with side opening 1830. Once aligned, rotation of delivery sheath 1820 relative to heart valve prosthesis 100 allows sequential lateral release of engagement arms 122 through respective side opening 1830 such that each of the engagement arms 122 returns to its flared configuration in a gradual controlled manner one after the other.

In the embodiment of FIG. 19, delivery sheath 1920 includes three side openings 1830 that are shaped to substantially match the rounded profile of proximal ends 124 of engagement arms 122 of heart valve prosthesis 100. Each of side openings 1830 includes an open distal end portion 1864 as described with reference to the embodiment of FIG. 18. With reference to deployment of heart valve prosthesis 100 shown in FIG. 1, heart valve prosthesis 100 is longitudinally translated relative to delivery sheath 1920 to laterally align proximal ends 124 of engagement arms 122 with side openings 1830. Once aligned, rotation of delivery sheath 1920 relative to heart valve prosthesis 100 allows simultaneous gradual release of engagement arms 122 laterally through respective side openings 1830 such that engagement arms 122 return to their flared configuration in a controlled manner at the same time.

External edges of the side openings or slots discussed in the preceding embodiments may have chamfered external edges to avoid unintentionally catching internal cardiac or other structures during use in deploying a heart valve prosthesis in vivo.

While various embodiments have been described above, it should be understood that they have been presented only as illustrations and examples of the present invention, and not by way of limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A delivery sheath for controlling deployment of a self-expanding support structure of a prosthetic valve that flares in a proximal direction upon implantation in vivo comprising:
a tubular body portion defining a delivery lumen between a proximal end and a distal end thereof; and
a side opening formed through a side wall of the tubular body portion, wherein the side opening proximally extends from the distal end of the delivery sheath within the side wall.

2. The delivery sheath of claim 1, wherein the tubular body portion includes a plurality of side openings that proximally extend from the distal end of the delivery sheath within the side wall of the tubular body portion.

3. The delivery sheath of claim 2, wherein the tubular body portion includes a distal segment of a first material and a proximal segment of a second material and the plurality of side openings are formed through a side wall of the distal segment.

4. The delivery sheath of claim 3, wherein the first material is a metal and the second material is a polymer.

5. The delivery sheath of claim 1, wherein the side opening includes a right triangle shaped portion that is defined within the side wall of the tubular body portion by a side hypotenuse segment, a side leg segment and a base leg segment and a narrow channel portion that proximally extends within the side wall of the tubular body portion between the delivery sheath distal end and the right triangle shaped portion.

6. The delivery sheath of claim 5, wherein the side hypotenuse segment and the side leg segment distally extend toward each other from the base leg segment and are spaced from each other by the narrow channel portion.

7. The delivery sheath of claim 1, wherein the side opening is a spiral channel that winds around the tubular body portion from an open distal end at the delivery sheath distal end to a closed proximal end.

8. The delivery sheath of claim 1, wherein the tubular body portion includes a distal segment having a crown-shape with bulbous-topped projections such that a plurality of side openings are defined between adjacent bulbous-topped projections.

9. The delivery sheath of claim 1, wherein the side opening has a shape similar to one of a rectangle, square, wedge, wave, or quadrant.

10. The delivery sheath of claim 9, wherein the side opening is spaced from the distal end of the tubular body portion by a narrow channel.

11. The delivery sheath of claim 1, wherein the side opening has a shape similar to a profile of the self-expanding support structure of the prosthetic valve.

12. A method of controlling deployment of a self-expanding support structure of a prosthetic valve that flares in a proximal direction upon implantation in vivo comprising:
advancing the prosthetic valve with the self-expanding support structure within a lumen of a delivery sheath such that the self-expanding support structure is held in a compressed delivery configuration within the delivery sheath lumen;
aligning the self-expanding support structure of the prosthetic valve with a side opening of the delivery sheath, wherein the side opening proximally extends within a side wall of the delivery sheath from a distal end thereof; and
rotating the prosthetic valve relative to the delivery sheath whereby the self-expanding support structure is laterally released from the delivery sheath lumen through the side opening in the delivery sheath to gradually return from the compressed delivery configuration to a proximally flared deployed configuration in a controlled manner.

13. The method of claim 12, wherein the prosthetic valve includes a plurality of self-expanding support structures which are consecutively released from the delivery sheath lumen through the side opening.

14. The method of claim 12, wherein the delivery sheath includes a plurality of side openings that proximally extend within the delivery sheath side wall from the distal end thereof.

15. The method of claim 14, wherein the prosthetic valve includes a plurality of self-expanding support structures each of which is laterally released from the delivery sheath lumen through a respective side opening.

16. The method of claim 15, wherein the step of aligning the self-expanding support structures with the delivery sheath side openings permits the self-expanding support structures to be partially released from the side openings via relative longitudinal movement between the prosthetic valve and the delivery sheath.

17. The method of claim 16, wherein reverse relative longitudinal movement between the prosthetic valve and the delivery sheath recaptures the partially released self-expanding support structures within the delivery sheath lumen.

18. A method of implanting a heart valve prosthesis having a self-expanding engagement arm that in a deployed configuration flares in a proximal direction comprising:
gaining access to a ventricle of the heart;
advancing a guidewire through the ventricle and across a heart valve to be replaced;
advancing a delivery sheath along the guidewire to a treatment site across the heart valve, wherein the delivery sheath has a tubular body portion that defines a delivery sheath lumen and wherein a side opening proximally extends through a side wall of the tubular body portion from a distal end of the delivery sheath;
advancing a heart valve prosthesis through the delivery sheath lumen until the heart valve prosthesis is positioned for deployment at the treatment site, wherein the self-expanding engagement arm of the heart valve prosthesis is held in a compressed delivery configuration within the delivery sheath lumen;
distally advancing the heart valve prosthesis relative to the delivery sheath to align the self-expanding engagement arm with the side opening of the delivery sheath; and
rotating the heart valve prosthesis relative to the delivery sheath to gradually slide the engagement arm through the delivery sheath side opening whereby the engagement arm transitions in a controlled manner from the compressed delivery configuration to the proximally flared deployed configuration.

19. The method of claim 18, wherein the heart valve prosthesis includes a plurality of self-expanding engagement arms which are consecutively released from the delivery sheath lumen through the side opening as the heart valve prosthesis is rotated relative to the delivery sheath.

20. The method of claim 18, wherein the delivery sheath includes a plurality of side openings that proximally extend within the side wall of the tubular body portion from the distal end thereof.

21. The method of claim 20, wherein the heart valve prosthesis includes a plurality of self-expanding engagement arms each of which is laterally released from the delivery sheath lumen through a respective side opening.

22. The method of claim 21, wherein the step of aligning the self-expanding engagement arms with the delivery sheath side openings permits the self-expanding engagement arms to be partially released from the side openings via relative longitudinal movement between the heart valve prosthesis and the delivery sheath.

23. The method of claim 22, wherein reverse relative longitudinal movement between the heart valve prosthesis and the delivery sheath recaptures the partially released self-expanding engagement arms within the delivery sheath lumen.
